(19) United States
(12) Patent Application Publication
(10) Pub. No.: US 2019/0175378 A1
(43) Pub. Date: Jun. 13, 2019

(54) ELASTIC BAND FOR CORRECTING JOINT DEFORMITY AND ENHANCING JOINT FUNCTION, AND DEVICE FOR MOUNTING SAME

(71) Applicant: AJOU UNIVERSITY INDUSTRY-ACADEMIC COOPERATION FOUNDATION, Buwon-si, Gyeonggi-do (KR)

(72) Inventor: Ji Man HONG, Yongin-si, Gyeonggi-do (KR)

(73) Assignee: AJOU UNIVERSITY INDUSTRY-ACADEMIC COOPERATION FOUNDATION, Suwon-si, Gyeonggi-do (KR)

(21) Appl. No.: 16/321,041

(22) PCT Filed: Jul. 13, 2017
(86) PCT No.: PCT/KR2017/007520

§ 371 Date: Jan. 28, 2019

(30) Foreign Application Priority Data
Jul. 28, 2016 (KR) 10-2016-0095881

(51) Int. Cl. A61F 5/01

(52) U.S. Cl. CPC A61F 5/0113; A61F 5/0118

(57) ABSTRACT

Provided is an elastic band for correcting a joint deformity and enhancing a joint function, the elastic band including: a first band to be attached to an outer part of a joint of a finger or toe and to the back of a hand or the top of a foot, and having a strip shape; a second band to be attached to an inner part of a last joint of the finger or toe while surrounding the inner part, and having a width increasing towards an end to surround side parts of the finger or toe together; and a connecting portion disposed between the first band and the second band to integrally connect the first band and the second band, and having a width gradually decreasing and then increasing from the first band towards the second band, and a mounting device of the elastic band.

### TECHNICAL FIELD

The present disclosure relates to an elastic band for correcting a joint deformity and enhancing a joint function, and a mounting device of the elastic band, and more particularly, to an elastic band for correcting a joint deformity and enhancing a joint function, which corrects and strengthens a joint position of a patient whose joint of an upper or lower limb is bent due to a stroke or a nervous disorder, and a mounting device of the elastic band.

### BACKGROUND ART

In general, a patient with a stroke or a disorder in a central nervous system may have a dysfunction in the upper limb or a flexion deformity in the wrist due to strengthened flexor muscle in the hand or arm, together with motor disturbance. There is also a risk of equinus in the foot or ankle of the patient or varus where the foot is bent inward.

Such a deformation in the body causes inconvenience in the patient's social life in terms of functions and aesthetics, and in particular, the patient may be unable to walk due to a deformation of the leg and may lose a gripping ability due to a deformation of the arm.

In order to correct such a disorder, the patient is recommended to use a hard and strong assistive device to spread the bent joint of the patient and to receive rehabilitation.

However, since such orthosis is formed of a strong material that does not deform, the skin may be damaged, and since it is inconvenient to wear a glove or shoe outside the orthosis after the orthosis is mounted, the patient needs outside help to wear the glove or shoe.

In order to solve such problems, orthoses that are formed of a flexible material and are easily self-worn by patients by being customized to the patients' body shapes have been developed, and JP 2016-096892 discloses a related technology.

Here, a glove for training of bending and straightening fingers of a rehabilitated person is provided, wherein right or left two gloves are laminated and fixed vertically; the fingers of the rehabilitated person or a practitioner are inserted in one glove, and the fingers of the practitioner or the rehabilitated person are inserted in the other glove; and the fingers of the rehabilitated person side and the fingers of the practitioner side are vertically fixed, correspondingly and respectively.

However, it is still inconvenient for a patient who has low motor skills to self-wear a conventional orthosis, and it is difficult to hygienically manage the orthosis despite the orthosis is repeatedly used, and thus bacterial infection may occur in a region contacting the body.

### DESCRIPTION OF EMBODIMENTS

### Technical Problem

The present disclosure is designed to solve the problems of the related art, and therefore the present disclosure is directed to providing an elastic band for correcting a joint deformity and enhancing a joint function, which may be easily self-mounted by a wearer and hygienically used, and a mounting device therefor.

### Solution To Problem

According to an aspect of the present disclosure, an elastic band for correcting a joint deformity and enhancing a joint function, the elastic band includes: a first band to be attached to an outer part of a joint of a finger or toe and to the back of a hand or the top of a foot, and having a strip shape; a second band to be attached to an inner part of a last joint of the finger or toe while surrounding the inner part, and having a width increasing towards an end to surround side parts of the finger or toe together; and a connecting portion disposed between the first band and the second band to integrally connect the first band and the second band, and having a width gradually decreasing and then increasing from the first band towards the second band, wherein the elastic band is elastic has a bottom surface on which an adhesive is coated to be attached to at least a part of a finger, the back of a hand, and a wrist or at least a part of a toe, the top of a foot, and an ankle of a wearer to correct deformed joints of the finger and wrist or deformed joints of the toe and ankle and enhance a function.

The elastic band may further include a cushioning member attached to a bottom surface of the connecting portion to relieve the end of the finger or toe from being pressed.

According to another aspect of the present disclosure, a mounting device of an elastic band for correcting a joint deformity and enhancing a joint function, the mounting device includes: a first body having a top surface whose shape corresponds to a palm or sole to accommodate the palm or sole; a second body having a top surface whose shape is curved downward correspondingly to a finger or toe to accommodate an end of the finger or toe; and an elastic portion integrally connecting the first body and the second body and providing elasticity to spread a bent finger or toe, wherein the elastic band for correcting a deformity of a deformed joint of a finger and wrist or a deformed joint of a toe and ankle and enhancing a function is mounted on the mounting device.

The elastic portion may have a width relatively smaller than that of the second body and have a shape curved downward.

The mounting device may further include a fixing belt fixing a hand or foot while the finger or toe is spread.

The mounting device may further include a first Velcro portion provided with Velcro for fixing a top surface of one end of the elastic band to a portion of the second body corresponding to each finger or toe.

The mounting device may further include a second Velcro provided with Velcro that is provided at a bottom surface of the first body to fix the first body.

The mounting device may further include a handle portion provided at a rear end of the first body.

The mounting device may further include, when the elastic band is mounted on a lower limb, a third Velcro portion provided with Velcro that is provided at a top surface of the first body to fix an end of a valgus correction band for surrounding an ankle.

### Advantageous Effects of Disclosure

Following effects may be obtained according to an elastic band for correcting a joint deformity and enhancing a joint function, and a mounting device of the elastic band.

First, the elastic band may be manufactured to be disposable, and thus hygiene may be maintained while the elastic band is used and bacterial infection caused by contamination of the elastic band may be prevented.

Second, while a wearer (patient) mounts the elastic band on his or her lower limb or upper limb, the patient's bent finger or toe is spread and fixed by using the mounting device assisting the elastic band, and thus the patient is able to self-mount the elastic band on the upper limb or lower limb, and attachment of the elastic band and correction of the joint may be possible with a more accurate position.

Third, with respect to the patient who is unable to grip an object due to bending between the fingers and the wrist, the patient's fingers and wrist may be spread to assist a gripping motion of the patient.

Fourth, with respect to the patient who has equinus or varus caused by bending between the toes and the ankle, the positions of the toes and ankle may be corrected to assist walking of the patient.

### BRIEF DESCRIPTION OF DRAWINGS

FIG. 1 is a perspective view showing a lower portion of an elastic band for correcting a joint deformity and enhancing a joint function, according to an embodiment of the present disclosure.

FIG. 2 is a use state diagram showing a state in which the elastic band of FIG. 1 is mounted on an upper limb.

FIG. 3 is a use state diagram showing a state in which the elastic band of FIG. 1 is mounted on a lower limb.

FIG. 4 is a perspective view showing a mounting device for mounting the elastic band of FIG. 1 on an upper limb.

FIG. 5 is a use state diagram showing a state in which an upper limb position of a wearer is fixed by using the mounting device of FIG. 4.

FIG. 6 is a perspective view showing a mounting device for mounting the elastic band of FIG. 1 on a lower limb.

FIG. 7 is a use state diagram showing a state in which a lower limb position of a wearer is fixed by using the mounting device of FIG. 6.

### BEST MODE

Hereinafter, the present disclosure will be described more fully with reference to the accompanying drawings, in which exemplary embodiments of the disclosure are shown. The terms or words used herein must not be interpreted in their common or dictionary definitions, but must be interpreted in the meanings and concept corresponding to the aspect of the present disclosure, based on the principle that the inventor(s) can suitably define the concept of terms in order to describe the disclosure in the best manner.

Accordingly, the embodiments and drawings described herein are only preferred examples, and do not represent the technical aspects of the present disclosure. Thus, one of ordinary skill in the art understands that the disclosure may be embodied in many different forms.

Hereinafter, exemplary embodiments of the present disclosure will be described in detail with reference to accompanying drawings.

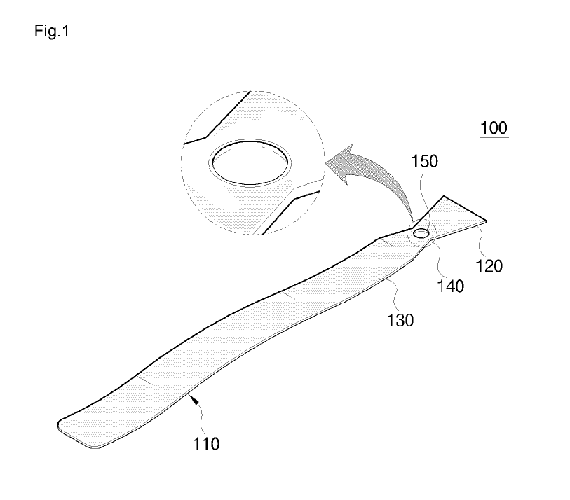

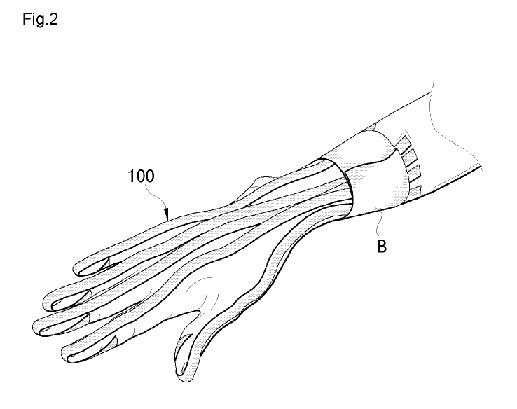

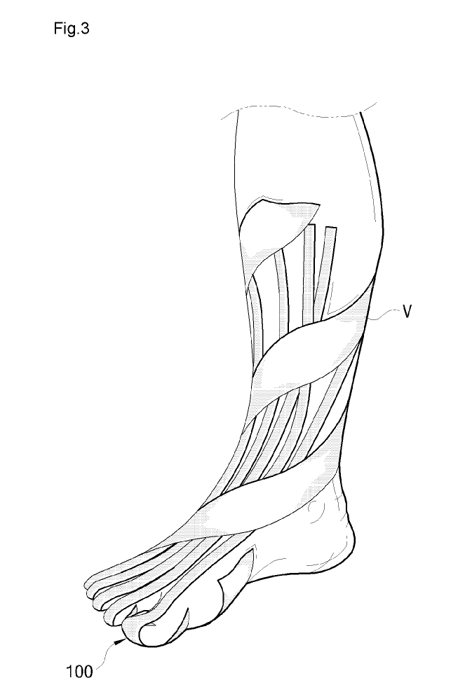

FIG. 1 is a perspective view showing a lower portion of an elastic band for correcting a joint deformity and enhancing a joint function, according to an embodiment of the present disclosure, FIG. 2 is a use state diagram showing a state in which the elastic band of FIG. 1 is mounted on an upper limb, and FIG. 3 is a use state diagram showing a state in which the elastic band of FIG. 1 is mounted on a lower limb.

Referring to the drawings, an elastic band for correcting a joint deformity and enhancing a joint function 100 (hereinafter, referred to as an “elastic band”), according to an embodiment of the present disclosure is for correcting a joint position by being attached to surround at least a part of an upper limb or lower limb and pulling a joint of the upper limb or lower limb. In this regard, the elastic band 100 includes a first band 110, a second band 120, and a connecting portion 130. The elastic band 100 is formed of an elastic material and an adhesive is coated on a bottom surface thereof. Here, the adhesive is protected by coating a release paper on the adhesive, but the release paper is not shown in the drawings. Meanwhile, the attached drawings only illustrate the elastic band 100 being used for the upper limb or lower limb, but it would be obvious that the elastic band 100 may be used to correct a position of another joint by being applied to another body part.

The first band 110 is attached to an outer part of a joint of a finger or toe and to the back of a hand or the top of a foot, and has a strip shape. Here, the first band has tension and elastically pulls the finger or toe.

The second band 120 surrounds an inner part of the last joint of the finger or toe. Here, the width of the second band 120 increases towards an end to surround side parts of the finger or toe together. A portion of the second band 120 whose width is increased as such surrounds the side parts of the finger or toe such that the second band 120 is stably fixed and a wearing sensation felt a wearer (patient) may be improved.

The connecting portion 130 is disposed between the first band 110 and the second band 120 to integrally connect the first and second bands 110 and 120. Here, since the connecting portion 130 is bendably attached to an end of the finger or toe as shown in FIG. 2 or 3, the connecting portion 130 has the width gradually decreasing and then increasing from the first band 110 towards the second band 120 to match such an attaching shape.

In the above embodiment of the present disclosure, the elastic band 100 may include a cushioning member 140 to relieve the end of the finger or toe of the wearer from being pressed. In this regard, the cushioning member 140 is attached to a bottom surface of the connecting portion 130 on which the adhesive is coated. Here, the cushioning member 140 relieves the end of the finger or toe from being excessively pressed to the connecting portion 130 due to tension of the first and second bands 110 and 120, and may be configured in a form of a cushion, but is not limited thereto.

As shown in FIGS. 2 and 3, the elastic band 100 corrects a joint position of the finger or toe by being attached to each finger or toe of an upper limb or lower limb of the wearer. Here, as shown in FIG. 2, when the elastic band 100 is attached to the upper limb of the wearer, the plurality of elastic bands 100 may be respectively attached to the fingers of the wearer, and then ends of the elastic bands 100 may be fixed such as not to be detached by surrounding a fixing band B in a tape form around an arm.

Meanwhile, as shown in FIG. 3, when the elastic band 100 is attached to the lower limb of the wearer, the plurality of elastic bands 100 may be respectively attached to the toes of the wearer, and then a valgus correction band V winding around the sole, ankle, and calf of the wearer may be additionally attached to fix the end of the elastic band 100 while correcting valgus fracture of the joint of the ankle of the wearer (patient).

Such an elastic band 100 according to the present disclosure may assist the patient to perform a gripping motion by allowing the finger and wrist of the patient to be spread with respect to the patient who is unable to grip an object due to a bending between the finger and wrist, and may assist the patient to walk by correcting positions of the two and ankle with respect to the patient with equinus or varus occurred due to bending between the toe and ankle. Moreover, the elastic band 100 may be manufactured to be disposable such that hygiene is maintained while the elastic band 100 is used and bacterial infection caused by contamination of the elastic band 100 is prevented.

Meanwhile, according to another aspect of the present disclosure, mounting devices of an elastic band 200 or 300 (hereinafter, referred to as “mounting devices”) for correcting a position of a joint of a finger or toe and fixing motion of an upper limb or lower limb of a wearer while the elastic band 100 is attached to the upper limb or lower limb are provided.

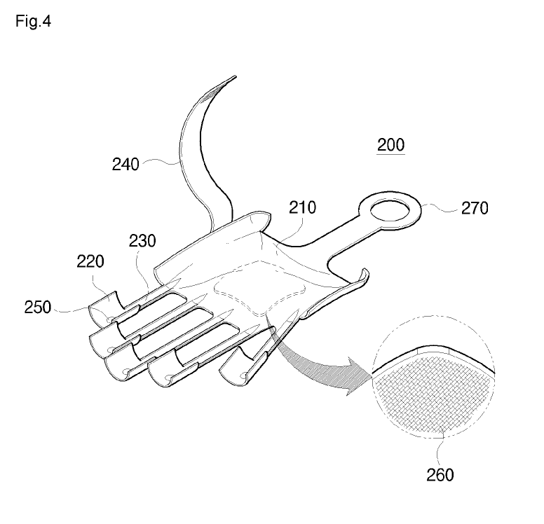

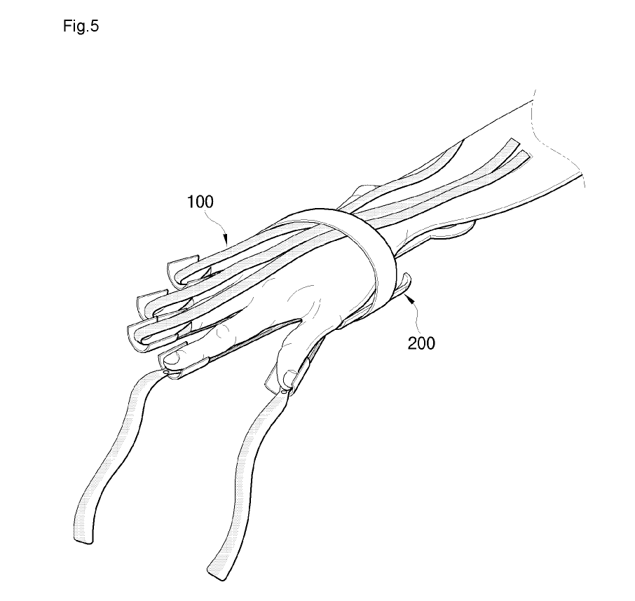

Hereinafter, a structure and method of using the mounting device 200 according to an embodiment of the present disclosure will be described with reference to FIGS. 4 and 5. Here, the mounting device 200 is a device assisting mounting of the elastic band 100 by fixing a position of a bent upper limb of a wearer while the wearer mounts the elastic band 100 on an upper limb part.

FIG. 4 is a perspective view showing a mounting device for mounting the elastic band of FIG. 1 on an upper limb, and FIG. 5 is a use state diagram showing a state in which an upper limb position of a wearer is fixed by using the mounting device of FIG. 4.

Referring to the drawings, the mounting device 200 according to an embodiment of the present disclosure is for assisting mounting of the elastic band 100 on an upper limb part of a wearer, and includes a first body 210, a second body 220, and an elastic portion 230.

A palm of the wearer is mounted on the first body 210, and a top surface of the first body 210 has a shape corresponding to the palm. Here, the wearer manipulates the first body 210 such that the first body 210 moves forward to slide while contacting the palm of the wearer.

The second body 220 has a top surface whose shape is curved downward correspondingly to the finger such that an end of the finger is accommodated. Here, the second body 220 performs a function of pushing and spreading a bent finger of the wearer via the sliding of the first body 210. Also, side ends of the second body 220 having a downward curved shape may prevent the finger of the wearer from being deviated. Here, a first Velcro portion 250 described later may be provided at the top surface of the second body 220 such that one end of the elastic band 100 is temporarily fixed to the top surface of the second body 220.

The elastic portion 230 connects the first body 210 and the second body 220, and is formed of an elastic material. Here, the elastic portion 230 has a width relatively smaller than that of the second body 220 such that the accommodated finger is gradually spread by an elastic restoring force by bending. Here, since the elastic portion 230 has the downward curved shape, the elastic portion 230 is able to not only provide a sufficient elastic restoring force, but also prevent deformation by bending or the like.

Meanwhile, the mounting device 200 may further include a fixing belt 240 to fix the hand of the wearer to the first body 210 while the finger is spread. In such a fixing belt 240, one end extends from the side end of the first body 210 and another end is attached to or detached from the other side end of the first body 210. Here, the fixing belt 240 may include detachable Velcro at the end.

Also, the mounting device 200 may include the first Velcro portion 250 to temporarily fix the elastic band 100 to the second body 220. The first Velcro portion 250 is provided at a portion of the second body 220 corresponding to each finger, and includes Velcro for fixing the top surface of one end of the elastic band 100. Here, the first Velcro portion 250 is arranged such that the top surface of the second band 120 without adhesive strength is fixed and the bottom surface of the second band 120 having adhesive strength faces the hand to be accommodated. In this case, the elastic band 100 temporarily fixed to the first Velcro portion 250 may be attached to the hand of the wearer when the finger of the wearer is accommodated on the second body 220 and an inner part of the last joint of the finger naturally contacts an adhesive surface of the second band 120.

In addition, the mounting device 200 may further include a second Velcro portion 260 provided at the bottom surface of the first body 210 and fixing the first body 210 to a fixing target. The fixing target may include a cloth material, such as fiber, so as to be detachable by Velcro, but is not limited thereto. Here, the wearer may perform an attaching process in a more stable position by attaching the first band 110 of the elastic band 100 to the back of the finger and the back of the hand while the hand is fixed to the fixing target together with the first body 210.

Meanwhile, the mounting device 200 may include a handle portion 270 extending from a rear end of the first body 210. Here, the wearer slides the first body 210 forward by holding the handle portion 270 with the other hand on which the elastic band 100 is not mounted.

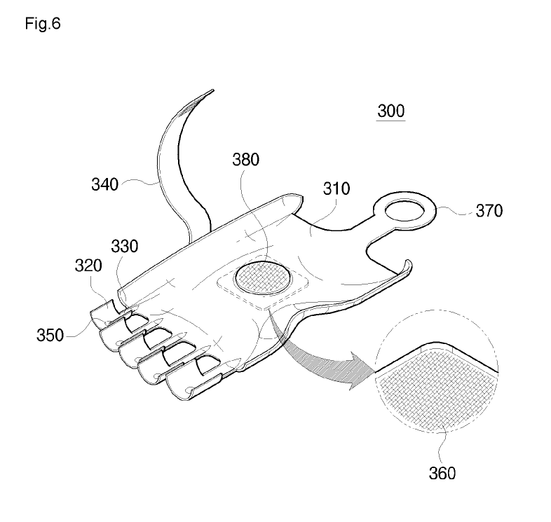

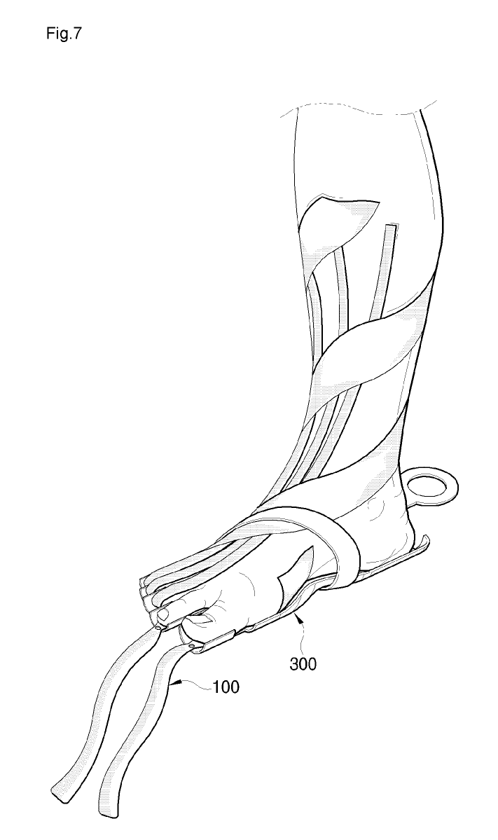

Hereinafter, a structure and method of using the mounting device 300 according to another embodiment of the present disclosure will be described with reference to FIGS. 6 and 7. Here, the mounting device 300 is a device assisting mounting of the elastic band 100 by fixing a position of a bent upper limb of a wearer while the wearer mounts the elastic band 100 on a lower limb part.

FIG. 6 is a perspective view showing a mounting device for mounting the elastic band of FIG. 1 on a lower limb, and FIG. 7 is a use state diagram showing a state in which a lower limb position of a wearer is fixed by using the mounting device of FIG. 6.

Referring to the drawings, the mounting device 300 according to another embodiment of the present disclosure assists mounting of the elastic band 100 on a lower limb portion of a wearer, and includes a first body 310, a second body 320, and an elastic portion 330.

A sole of a foot is mounted on the first body 310, and a top surface of the first body 310 has a shape corresponding to the sole. Here, the wearer manipulates the first body 310 such that the first body 310 moves forward to slide while contacting the sole of the wearer.

The second body 320 has a top surface whose shape is curved downward correspondingly to the toe such that an end of the toe is accommodated. Here, the second body 320 performs a function of pushing and spreading a bent toe of the wearer via the sliding of the first body 310. Also, side ends of the second body 320 having a downward curved shape may prevent the toe of the wearer from being deviated. Here, a first Velcro portion 350 described later may be provided at the top surface of the second body 320 such that the elastic band 100 is temporarily fixed to the top surface of the second body 320.

The elastic portion 330 connects the first body 310 and the second body 320, and is formed of an elastic material. Here, the elastic portion 330 has a width relatively smaller than that of the second body 320 such that the accommodated toe is gradually spread by an elastic restoring force by bending. Here, since the elastic portion 330 has the downward curved shape, the elastic portion 330 is able to not only provide a sufficient elastic restoring force, but also prevent deformation by bending or the like.

Meanwhile, the mounting device 300 may further include a fixing belt 340 to fix the foot of the wearer to the first body 310 while the toe is spread. In such a fixing belt 340, one end extends from the side end of the first body 310 and another end is attached to or detached from the other side end of the first body 310. Here, the fixing belt 340 may include detachable Velcro at the end.

Also, the mounting device 300 may include the first Velcro portion 350 to temporarily fix the elastic band 100 to the second body 320. The first Velcro portion 350 is provided at a portion of the second body 320 corresponding to each toe, and includes Velcro for fixing the top surface of one end of the elastic band 100. Here, the first Velcro portion 350 is arranged such that the top surface of the second band 120 without adhesive strength is fixed and the bottom surface of the second band 120 having adhesive strength faces the foot to be accommodated. In this case, the elastic band 100 temporarily fixed to the first Velcro portion 350 may be attached to the foot of the wearer when the toe of the wearer is accommodated on the second body 320 and an inner part of the last joint of the toe naturally contacts an adhesive surface of the second band 120.

In addition, the mounting device 200 may further include a second Velcro portion 360 provided at the bottom surface of the first body 310 and fixing the first body 310 to a fixing target. The fixing target may be cloth, such as a fiber material, but is not limited thereto. Here, the wearer may attach the first band 110 of the elastic band 100 to the top of the toe and the top of the foot while the foot is fixed to the fixing target together with the first body 310.

Also, the mounting device 300 of the present disclosure may further include a handle portion 370 extending from a rear end of the first body 310. Here, the wearer slides the first body 310 forward by holding the handle portion 370 with the hand.

Meanwhile, the mounting device 300 may further include a third Velcro portion 380 to fix an end of the valgus correction band V for surrounding the ankle of the wearer. The third Velcro portion 380 includes Velcro provided at the top surface of the first body 310. In detail, the valgus correction band V corrects a valgus fracture of the joint of the ankle of the wearer by surrounding the ankle and calf of the wearer. Here, the valgus correction band V may generally have a wider width and stronger tension than the elastic band 100. The third Velcro portion 380 is adhered to an end of the valgus correction band V without adhesive strength. In this case, when the wearer accommodates the foot on the top surface of the first body 310, the center of the sole of the wearer contacts a surface of the valgus correction band V having adhesive strength and the end of the valgus correction band V is naturally attached to the sole of the wearer.

As described above, according to the mounting devices 200 and 300 of the present disclosure, while a wearer (patient) mounts an adhesive elastic band on his/her upper limb or lower limb, the mounting is assisted to spread and fix the bent finger or toe of the patient such that the patient is able to self-mount the elastic band on the upper limb or lower limb, and the elastic band is attached and the joint is corrected in a more accurate position.

While the present disclosure has been particularly shown and described with reference to exemplary embodiments thereof, it will be understood by those of ordinary skill in the art that various changes in form and details may be made therein without departing from the spirit and scope of the present disclosure as defined by the following claims.

1. An elastic band for correcting a joint deformity and enhancing a joint function, the elastic band comprising:
a first band to be attached to an outer part of a joint of a finger or toe and to the back of a hand or the top of a foot, and having a strip shape;
a second band to be attached to an inner part of a last joint of the finger or toe while surrounding the inner part, and having a width increasing towards an end to surround side parts of the finger or toe together; and
a connecting portion disposed between the first band and the second band to integrally connect the first band and the second band, and having a width gradually decreasing and then increasing from the first band towards the second band,
wherein the elastic band is elastic and has a bottom surface on which an adhesive is coated to be attached to at least a part of a finger, the back of a hand, and a wrist or at least a part of a toe, the top of a foot, and an ankle of a wearer to correct deformed joints of the finger and wrist or deformed joints of the toe and ankle and enhance a function.

2. The elastic band of claim 1, further comprising a cushioning member attached to a bottom surface of the connecting portion to relieve the end of the finger or toe from being pressed.

3. A mounting device of an elastic band for correcting a joint deformity and enhancing a joint function, the mounting device comprising:
a first body having a top surface whose shape corresponds to a palm or sole to accommodate the palm or sole;
a second body having a top surface whose shape is curved downward correspondingly to a finger or toe to accommodate an end of the finger or toe; and
an elastic portion integrally connecting the first body and the second body and providing elasticity to spread a bent finger or toe,
wherein the elastic band for correcting a deformity of a deformed joint of a finger and wrist or a deformed joint of a toe and ankle and enhancing a function is mounted on the mounting device.

4. The mounting device of claim 3, wherein the elastic portion has a width relatively smaller than that of the second body and has a shape curved downward.

5. The mounting device of claim 3, further comprising a fixing belt fixing a hand or foot while the finger or toe is spread.

6. The mounting device of claim 3, further comprising a first Velcro portion provided with Velcro for fixing a top surface of one end of the elastic band to a portion of the second body corresponding to each finger or toe.

7. The mounting device of claim 3, further comprising a second Velcro provided with Velcro that is provided at a bottom surface of the first body to fix the first body.

8. The mounting device of claim 3, further comprising a handle portion provided at a rear end of the first body.

9. The mounting device of claim 3, further comprising, when the elastic band is mounted on a lower limb, a third Velcro portion provided with Velcro that is provided at a top surface of the first body to fix an end of a valgus correction band for surrounding an ankle.
